(19) United States
(12) Patent Application Publication
(10) Pub. No.: US 2010/0031380 A1
(43) Pub. Date: Feb. 4, 2010

(54) IDENTIFICATION OF CHILLING-RESISTANT PLANTS

(75) Inventors: Beate Kamlage, Berlin (DE); Oliver Bläsing, Potsdam (DE); Yves Gibon, Berlin (DE); Mark Stitt, Potsdam (DE)

Correspondence Address: CONNOLLY BOVE LODGE & HUTZ, LLP, P O BOX 2207, WILMINGTON, DE, 19899, US

(73) Assignee: METANOMICS GMBH, Berlin (DE)

(21) Appl. No.: 12/525,351

(22) PCT Filed: Feb. 6, 2008
(86) PCT No.: PCT/GB2008/000419

§ 371 Date: Jul. 31, 2009

(30) Foreign Application Priority Data
Feb. 6, 2007 (GB) 0702262.7

(51) Int. Cl. A01H 1/00; C12Q 1/02

(52) U.S. Cl. 800/260; 435/29

(57) ABSTRACT

A process for identifying whether plant is resistant to chilling comprises cultivating the plant at a reduced temperature for a time period selected from short term and medium term; harvesting a tissue sample of the plant; measuring concentration of at least one metabolite in the tissue sample of the plant; comparing the measured concentration with the concentration of the same metabolite in a tissue sample harvested from a reference plant or a plant of the same species after cultivation at a reference temperature; and optionally, repeating the process of foregoing steps but cultivating the plant at a reduced temperature for a time period selected from short term and medium term and not previously used; wherein resistance to chilling is indicated by one or more of the effects on metabolites listed in Table 2 or Table 4.

The present invention relates to a method of identifying or selecting plants which are resistant to chilling, in particular chilling at a temperature of between about 6 and 17° C.

Temperatures change within minutes to hours in the diurnal cycle, in hours to days as a result of changing weather, and over weeks to months as a result of seasonal changes. Low temperatures impinge on a plethora of biological processes. They inhibit almost all metabolic and cellular processes, with the typical Q₁₀ for protein-dependent catalysis lying between 2 and 3. They impact on membrane-based processes, because low temperatures alter the physical properties of lipids and reduce membrane fluidity. At temperatures below zero, there is the additional danger of ice formation. This typically takes place in the apoplast, leading withdrawal of water and dehydration of the symplast. The response of plants to low temperature is an important determinant of their ecological range. The problem of coping with low temperatures is exacerbated by the need to prolong the growing season beyond the short summer found at high latitudes or altitudes.

Adaptation to low temperature is divided into chilling tolerance, and freezing resistance. Chilling tolerance is typically found in species from temperate or boreal zones, and allows survival and growth at low but non-freezing temperatures. Species from tropical or subtropical zones often show wilting, chlorosis or necrosis, slowed growth and even death at temperatures as high as 10-12° C. Freezing resistance allows survival at subzero temperatures. It is promoted by a process termed cold-acclimation, which occurs at low but non-freezing temperatures and provides increased freezing tolerance at subzero temperatures. In addition, most species from temperate regions have life cycles that are adapted to seasonal changes of the temperature. For those plants, low temperatures may also play an important role in plant development through the process of stratification and vernalisation.

The molecular basis of freezing tolerance has been intensively researched in Arabidopsis. Physiological changes during cold acclimation include changes in lipid composition to increase membrane fluidity, expression of proteins that modify the physical characteristics of membranes, accumulation of compatible solutes like sucrose, raffinose and proline (Cook et al., 2004), detoxification of active oxygen species and altered leaf development to increase the levels of proteins involved in photosynthetic electron transport and carbon fixation. Some of these changes are specific for low temperature, and others also occur in response to dehydration, mechanical stress or the addition of abscisic acid.

Studies of Arabidopsis pho1 and pho2 mutants with decreased and elevated shoot Pi indicate that some of the changes in photosynthetic and carbohydrate metabolism may be modulated by changes of Pi. In some species, freezing tolerance is improved by addition of abscisic acid or salicylic acid.

Less is known about the molecular basis of chilling tolerance. Exposure of chilling-sensitive species to low temperatures inhibits photosynthesis and leads to photoinhibition. Small decreases of the temperature lead to a transient Pi-limitation of photosynthesis. This occurs because the sucrose and starch synthesis are inhibited more strongly than carbon fixation, resulting in the sequestration of Pi in phosphorylated intermediates, and is reversed by post-translational and transcriptional stimulation of sucrose synthesis. Chilling often delays leaf development and interferes with plastid biogenesis, leading to delayed greening, chlorosis and thickening or deformation of new leaves. Chilling temperatures inhibit respiration, phloem transport, and restrict the utilization of photoassimilate for growth. As a result, carbohydrates usually accumulate in chilled plants. A decrease of water conductivity in the roots leads to shoot wilting, unless the stomates close. Chilling temperatures also lead to leakage of ions across cell membranes including the release of calcium from internal pools into the cytosol and oxidative stress.

Chilling-tolerance is a major breeding trait because several major crops such as maize, bean, tomato, cucumber and potato are chilling-sensitive. Breeding of chilling tolerant crops will result in a better trait for stress tolerance and is expected to increase the traits for quality and yield of the respective crop.

However, the genetic and molecular basis of chilling responses is poorly understood. Although genetic diversity has been identified, for example from landraces and related species that grow at light altitudes, and is being introduced into breeding lines the genes responsible for the qualitative trait loci have not yet been identified.

Most molecular studies of low temperature responses have addressed freezing tolerance and used temperatures of 0-4° C. to induce cold-acclimation, instead of less extreme temperatures that might be more relevant for understanding the chilling response. There may be some overlap between the responses which have predominantly been investigated on the transcriptional level.

A better understanding the chilling response requires more information about the response of tolerant species to chilling temperatures. Provart et al. (2003) transferred 4-week old soil-grown Arabidopsis Col-0 from 20° C. to 13° C. and analysed the expression of 8000 genes two days later. Genes assigned to protein synthesis (including ribosomal proteins) and, to a lesser extent, energy and central carbon metabolism were overrepresented in the chilling-induced genes.

There was a poor correlation between the transcriptional response to chilling (Provart et al., 2003) and the responses in earlier studies in which Arabidopsis seedlings on nutrient medium were transferred to 4° C. for one day (Fowler & Thomashow, 2002; Kreps et al., 2002). For example, many genes that are induced by the CBF family members were reported to be unaffected or even decreased at 13° C. This might be due to the use different expression profiling technologies, or reflect a qualitative difference between the chilling and cold-acclimation responses, or differences in the induction or relaxation kinetics at moderate and near-freezing temperatures. It might also reflect difficulties in identifying a robust set of cold-responsive genes. There are differences between the sets of cold-sensitive genes in soil- and nutrient-medium grown plants (Vogel et al., 2005). In a recent study of the response of 22,000 genes after transfer of nutrient medium-grown Arabidopsis from 2° C. to 0° C. (Lee et al., 2005), only about a third of the genes that were short listed in earlier studies were independently confirmed as cold-responsive. In summary, the comparison of the transcriptional response to chilling temperatures and temperatures close to 0° C. suggest qualitative, quantitative and temporal differences between the two responses.

The present inventors have carried out an analysis of the metabolic response to an increasingly severe chilling treatment. Soil-grown Arabidopsis wild-types were transferred from 20° C. to 17, 14, 12, 10 or 8° C., harvested 6 and 78 h later, and subjected to metabolite profiling to characterise the changes in metabolism.

The results of the experiments carried out by the inventors demonstrate that metabolite concentration in the leaves of chill-resistant plants is affected by chilling and the present inventors have used these results to devise a method of identifying plants which are likely to show resistance or sensitivity to chilling and to identify metabolites which are associated with chilling resistance. Identifying the metabolites which characterise chilling resistance further allows the engineering or modification of chilling resistance in plants.

Therefore, in a first aspect of the present invention, there is provided a process for identifying whether plant is resistant to chilling, the process comprising:

a) cultivating the plant at a reduced temperature for a time period selected from short term and medium term;

b) harvesting a tissue sample of the plant;

c) measuring concentration of at least one metabolite in the tissue sample of the plant;

d) comparing the measured concentration with the concentration of the same metabolite in a tissue sample harvested from a reference plant or a plant of the same species after cultivation at a reference temperature; and

e) optionally repeating the process of steps (a) to (d) but cultivating the plant at a reduced temperature for a time period selected from short term and medium term and not previously used in step (a);
wherein resistance to chilling is indicated by one or more of the effects on metabolites listed in Table 2 or Table 4.

Advantageously, resistance to chilling is indicated by two or more of the effects listed in table 5 or table 6.

Preferably, resistance to chilling is determined by mass spectrometry and indicated by two or more of the effects on the metabolites set forth in Table 5.

In a further embodiment, resistance to chilling may be determined by methods other than mass spectrometry, and indicated by two or more of the effects on the metabolites set forth in Table 6.

Preferably, 3 or more of the effects on three or more metabolites in Table 5 or Table 6 are measured; advantageously, 4, 5, 6, 7, 8, 9, 10 or more effects on the corresponding number of metabolites.

In an advantageous embodiment, resistance to chilling is indicated by one or more of the effects listed in table 7 or table 8.

Preferably, resistance to chilling is determined by mass spectrometry and indicated by one or more of the effects on the metabolites set forth in Table 7.

In a further embodiment, resistance to chilling may be determined by methods other than mass spectrometry, and indicated by one or more of the effects on the metabolites set forth in Table 8.

Preferably, two or more of the effects on two or more metabolites in Table 7 or Table 8 are measured; advantageously, 3, 4, 5, 6, 7, 8, 9, 10 or more effects on the corresponding number of metabolites.

Advantageously, resistance to chilling is indicted by one or more of the effects set forth in Table 5 or Table 6 in combination with one or more of the effects set forth in Table 7 or Table 8.

Thus, the invention represents a simple method of determining whether or not a plant is likely to show resistance to chilling. This method is of particular use in plant breeding, particularly in producing chill-resistant varieties of crop plants selected from the group consisting of Asteraceae such as the genera Helianthus, Tagetes e.g. the species Helianthus annus [sunflower], Tagetes lucida, Tagetes erecta or Tagetes tenuifolia [Marigold], Brassicaceae such as the genera Brassica, Arabidopsis e.g. the species Brassica napus, Brassica rapa ssp. [canola, oilseed rape, turnip rape] or Arabidopsis thaliania. Fabaceae such as the genera Glycine e.g. the species Glycine max [soybean], Soja hispida. Linaceae such as the genera Linum e.g. the species Linum usitatissimum, [flax, linseed]; Poaceae such as the genera Hordeum, Secale, Avena, Sorghum, Oryza, Zea, Triticum e.g. the species Hordeum vulgare [barley]; Secale cereale [rye], Avena sativa, Avena fatua, Avena byzantina, Avena fatua var. sativa, Avena hybrida [oat], Sorghum bicolor [Sorghum, millet], Oryza sativa, Oryza latifolia [rice], Zea mays [corn, maize] Triticum aestivum, Tritictum durum, Triticum turgidum, Triticum hybernum, Triticum macha, Triticum sativum or Triticum vulgare [wheat, bread wheat, common wheat]; Solanaceae such as the genera Solanum, Lycopersicon e.g. the species Solanum tuberosum [potato], Lycopersicon esculentum, Lycopersicon lycopersicum, Lycopersicon pyriforme, Solanum integrifolium or Solanum lycopersicum [tomato]. Specifically preferred are tomato, potato, bean, maize or rice, soybean, canola, linseed.

The experiments set forth herein were carried out on chill-resistant Arabidopsis species, which is accepted as a representative species in plant biology.

In the present application, the term “chilling” refers to reducing the temperature at which the plant is cultivated to between 6° C. and 17° C.

Similarly, the term “reduced temperature” refers to a temperature of between 6° C. and 17° C.

A “reference growth temperature” or “standard growth temperature” is a temperature at which the plant grows normally. This may be an optimum temperature, which is ideal for the growth of the plant and at which the plant display the most favourable growth characteristics; or a temperature at which the plant grows within an acceptable range of variation from the optimum. In any event, the “reference temperature” is a temperature which is higher than the “reduced temperature”. Advantageously, such a temperature is between 18 and 26° C., preferably 20-22° C., and more preferably 20° C.

The term “short term” refers to a period of from 1 hour up to and including 12 hours, preferably from 4 hours to 10 hours and more preferably 5 to 7 hours, typically 6 hours.

The term “medium term” refers to a time period of more than 12 hours and up to and including 96 hours. Preferably, medium term is from 24 hours to 96 hours and more preferably from 48 to 84 hours, typically 72 to 80 hours.

The term “cultivating” refers to the growth of a plant under controlled or monitored conditions. Controlled or monitored conditions refers to the control or monitor of growth conditions including but not limiting, to the light regime, light intensity, light spectral composition, day-night-cycle, humidity, water supply, nutrient supply and growth medium. In a preferred embodiment the plants are cultivated in pots with a defined amount and type of soil in a growth chamber, for which the above parameters and especially the temperature is controlled.

The term “harvesting” refers to the collection of a tissue sample of the plant for the metabolic analysis. The tissue sample of the plant can either be a part of the plant, like a leaf or a tip of a leaf or the flower or the complete aerial part of a plant, depending of the size of the plant and the type of analysis. In a preferred embodiment the tissue sample is quickly taken, with minimal damage of the sampled plant material and frozen with liquid nitrogen in a few seconds, preferably within 20 seconds, even more preferred within 10 seconds. The quick deep freezing of the tissue sample is of special importance in order to avoid or at least limit metabolic changes in response to the method step of “harvesting”. In one embodiment the aerial part of a well grown Arabidopsis plant with a weight of about 200-400 mg is quickly cut and stored in liquid nitrogen.

The term “measuring concentration” refers to the identification and quantification of metabolites comprised by the tissue sample. The identification of metabolites refers to the exact determination of the chemical composition and structure of a metabolite. This can be achieved by the determination of one or more chemical and or physical properties of a metabolite. The person skilled in the art is familiar with a whole range of methods which allow the measurement of metabolites. Often these methods combine a separation step with a determination step. Suitable techniques for separation include all chromatographic separation techniques such as liquid chromatography (LC), high performance liquid chromatography (HPLC), gas chromatography (GC), thin layer chromatography, size exclusion or affinity chromatography. These techniques are well known in the art and can be applied by the person skilled in the art without further ado. Most preferably, LC and/or GC chromatographic techniques are used in the context of the present invention. Suitable devices for the determination of metabolites are well known in the art. Preferably, mass spectrometry is used in particular gas chromatography mass spectrometry (GC-MS), liquid chromatography mass spectrometry (LC-MS), direct infusion mass spectrometry or Fourier transform ion-cyclotrone-resonance mass spectrometry (FT-ICR-MS), capillary electrophoresis mass spectrometry (CE-MS), high-performance liquid chromatography coupled mass spectrometry (HPLC-MS), quadrupole mass spectrometry, any sequentially coupled mass spectrometry, such as MS-MS or MS-MS-MS, inductively coupled plasma mass spectrometry (ICP-MS), pyrolysis mass spectrometry (Py-MS), ion mobility mass spectrometry or time of flight mass spectrometry (TOF). Most preferably, LC-MS and/or GC-MS are used as described in examples. Said techniques are disclosed in, e.g., Nissen, Journal of Chromatography A, 703, 1995: 37-57, U.S. Pat. No. 4,540,884 or U.S. Pat. No. 5,397,894, the disclosure content of which is hereby incorporated by reference. For measuring concentration, the determination of metabolites occurs quantitatively or at least semiquantitatively meaning that the signal intensity during the determination of metabolites is used to determine the exact or at least the relative amount of the metabolite in the sample. As an alternative or in addition to mass spectrometry techniques, the following techniques may be used for compound determination: nuclear magnetic resonance (NMR), magnetic resonance imaging (MRI), Fourier transform infrared analysis (FT-IR), ultraviolet (UV) spectroscopy, refraction index (RI), fluorescent detection, radiochemical detection, electrochemical detection, light scattering (LS), dispersive Raman spectroscopy or flame ionisation detection (FID). These techniques are well known to the person skilled in the art and can be applied without further ado. Preferably the metabolites are extracted from the tissue sample before the step “measuring concentrations” is performed. In a preferred embodiment the metabolites are extracted from the tissue sample with organic or inorganic solvents or various mixtures thereof. Moreover, in the case of gas chromatography it is preferably envisaged that the compounds are derivatised prior to gas chromatography. Suitable techniques for derivatisation are well known in the art. Preferably, derivatisation in accordance with the present invention relates to methoxymation and trimethylsilylation of, preferably, polar compounds and transmethylation, methoxymation and trimethylsilylation of, preferably, non-polar (i.e. lipophilic) compounds. Details for derivatisation are described in the Examples below.

“Metabolite” refers to small molecule compounds, such as substrates for enzymes of metabolic pathways, intermediates of such pathways or the products obtained by a metabolic pathway. Metabolic pathways are well known in the art, may vary between species and can be taken from different standard text books or publications. Accordingly, small molecule compound metabolites are preferably composed of the following classes of compounds: alcohols, alkanes, alkenes, alkines, aromatic compounds, ketones, aldehydes, carboxylic acids, esters, amines, imines, amides, cyanides, amino acids, peptides, thiols, thioesters, phosphate esters, sulfate esters, thioethers, sulfoxides, ethers, or combinations or derivatives of the aforementioned compounds. The small molecules among the metabolites may be primary metabolites which are required for normal cellular function, organ function or plant development and growth or health. Moreover, small molecule metabolites further comprise secondary metabolites often having essential ecological function, e.g. metabolites which allow an organism to adapt to its environment especially to abiotic stresses or defend an organism against different types of biological stresses like for example plant pathogens.

The term “comparing” refers to assessing whether the results of the measuring concentration described herein above in detail, i.e. the results of the qualitative or quantitative determination of a metabolite, are identical or similar to results from a tissue sample of a reference plant or differ there from. A reference plant refers to a plant which is as identical as possible to the plant grown at reduced temperature despite the fact that the reference plant is grown at reference temperature, preferably at optimal or near optimal growth temperature. Optimal or near optimal growth temperature refers to such growth temperature which allow optimal plant growth and development and do not induce a stress response in plants. Stress responses in plant can be identified by various means, known to the person skilled in the art, preferably by the induction of stress responsive genes, which have been described for many different plants and stresses. In a preferred embodiment optimal growth temperature refers to a temperature of 18° C. to 26° C. The person skilled in the art is aware that the optimal growth temperature varies between different plant species and varieties In one embodiment in the case of Arabidopsis optimal growth temperature refer to a temperature of about 20° C.

The terms “Increase in concentration” or “Decrease in concentration” refer to increase or decrease in the measured concentration of at least one metabolite. In a preferred embodiment the increase in concentration or decrease in concentration at least 5% more preferably at least 10%.

The term “onie or more of the effects” refers in one embodiment to the situation that of the metabolites measured at least 40% show an affect as listed in the corresponding table, in a more preferred embodiment at least 50% of the metabolites measured show an effect as listed, in an even more preferred embodiment at least 60% of the metabolites measured show an effect as listed and in an even more preferred embodiment at least 70% or 80% or 90% or 95% or 99% of the metabolites measured show an effect as listed.

It should be understood that the greater the percentage of the metabolites measured which show the effect identified herein, the greater the likelihood that the plant in question is resistant to chilling.

It is greatly preferred that in the process of the invention, the concentration of at least five of the listed metabolites is measured and compared with the concentration in a plant cultivated at reference growth temperature. More preferably the concentration of at least ten, or, in increasing order of preference at least twenty, thirty or forty of the listed metabolites will be measured.

Although it is possible to make a judgement as to whether a plant is chill resistant using one of short term and medium term chilling, it is preferable to measure metabolite concentration in both time scales as this enables a more accurate conclusion to be reached. In any analysis, there is to be expected an overlap of the chilling responsive metabolites with other environmental and nutritional inputs to the assayed plant. Accordingly, the greater the number of measurements taken over the greater number of timescales, the more accurate the result which is to be expected.

The method of the invention also makes it possible to select candidate plants for use in breeding programs to produce chill resistant plants. Therefore, in a further aspect of the invention, there is provided a method for obtaining plants which are resistant to chilling, the method comprising:
a) cultivating a series of plants;
b) identifying those plants which have greatest resistance to chilling using a method as described in the foregoing aspects of the invention; and
c) crossing the chill-resistant plants identified in step (b) to obtain progeny which are resistant to chilling.

Suitable plants for use in the method are as specified above for the first aspect of the invention.

The invention will now be further described with reference to the following examples. Unless defined otherwise, all technical and scientific terms used herein have the same meaning as commonly understood by one of ordinary skill in the art (e.g., in cell culture, molecular genetics, nucleic acid chemistry, hybridisation techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al, Short Protocols in Molecular Biology (1999) 4^{th} Ed, John Wiley & Sons, Inc. which are incorporated herein by reference) and chemical methods.

### EXAMPLE 1

### Plant Growth and Sampling

Arabidopsis thaliana ecotype Col-0 was grown in 6 cm pots soil (GS90/Vermiculite 1:1; (Einheitserde GS90, Gebrüder Patzer, Sinntal-Jossa, Germany) in a 12 hrs light/12 hrs dark cycle, at a light intensity of 130 μmol/n-²s and at a constant temperature of 20° C. for 4 weeks, at which time flowering had not commenced. After 4 weeks ambient growth, Arabidopsis plants were simultaneously moved from 20° C. to 20, 17, 14, 12, 10 and 8° C., 4 hrs after the beginning of the light period. A first batch of plants was harvested 6 h later. A second batch of plants was kept for further 72 h under the same range of temperatures, and then harvested. Plants were harvested by transferring above ground grown rosettes into liquid nitrogen under ambient irradiance. 5 replicate samples, each containing 3 rosettes, were collected. Samples were powdered under liquid nitrogen and stored at −80° C. until its use.

### EXAMPLE 2

### Metabolite Profiling

### Metabolic Analysis of Transformed Plants

The modifications identified in accordance with the invention, in the content of above-described metabolites, were identified by the following procedures.

### Sampling and Storage of the Samples

Sampling was performed directly in the controlled-environment chamber. The plants were cut using small laboratory scissors, rapidly weighed on laboratory scales, transferred into a pre-cooled extraction sleeve and placed into an aluminum rack cooled by liquid nitrogen. If required, the extraction sleeves can be stored in the freezer at −80° C. The time elapsing between cutting the plant to freezing it in liquid nitrogen amounted to not more than 10 to 20 seconds.

### Lyophilization

During the experiment, care was taken that the plants either remained in the deep-frozen state (temperatures <−40° C.) or were freed from water by lyophilization until the first contact with solvents.

The aluminum rack with the plant samples in the extraction sleeves was placed into the pre-cooled (−40° C.) lyophilization facility. The initial temperature during the main drying phase was −35° C. and the pressure was 0.120 mbar. During the drying phase, the parameters were altered following a pressure and temperature program. The final temperature after 12 hours was +30° C. and the final pressure was 0.001 to 0.004 mbar. After the vacuum pump and the refrigerating machine had been switched off, the system was flushed with air (dried via a drying tube) or argon.

### Extraction

Immediately after the lyophilization apparatus had been flushed, the extraction sleeves with the lyophilized plant material were transferred into the 5 ml extraction cartridges of the ASE device (Accelerated Solvent Extractor ASE 200 with Solvent Controller and AutoASE software (DIONEX)).

The 24 sample positions of an ASE device (Accelerated Solvent Extractor ASE 200 with Solvent Controller and AutoASE software (DIONEX)) were filled with plant samples, including some samples for testing quality control.

The polar substances were extracted with approximately 10 ml of methanol/water (80/20, v/v) at T=70° C. and p=140 bar, 5 minutes heating-up phase, 1 minute static extraction. The more lipophilic substances were extracted with approximately 10 ml of methanol/dichloromethane (40/60, v/v) at T=70° C. and p=140 bar, 5 minute heating-up phase, 1 minute static extraction. The two solvent mixtures were extracted into the same glass tubes (centrifuge tubes, 50 ml, equipped with screw cap and pierceable septum for the ASE (DIONEX)).

The solution was treated with commercial available internal standards, such as ribitol, L-glycine-2,2-d₂, L-alanine-2,3,3,3-d₄, methionine-d₃, Arginine_(¹³C), Tryptophan-d₅, and α-methylglucopyranoside and methyl nonadecanoate, methyl undecanoate, methyl tridecanoate, methyl pentadecanoate, methyl nonacosanoate.

The total extract was treated with 8 ml of water. The solid residue of the plant sample and the extraction sleeve were discarded.

The extract was shaken and then centrifuged for 5 to 10 minutes at least 1 400 g in order to accelerate phase separation. 1 ml of the supernatant methanol/water phase (“polar phase”, colorless) was removed for the further GC analysis, and 1 ml was removed for the LC analysis. The remainder of the methanol/water phase was discarded. 0.5 ml of the organic phase (“lipid phase”, dark green) was removed for the further GC analysis and 0.5 ml was removed for the LC analysis. All the portions removed were evaporated to dryness using the IR Dancer infrared vacuum evaporator (Hettich). The maximum temperature during the evaporation process did not exceed 40° C. Pressure ill the apparatus was not less than 10 mbar.

### Processing the Lipid and Polar Phase for the LC/MS or LC/MS/MS Analysis

The lipid extract, which had been evaporated to dryness was taken up in mobile phase. The polar extract, which had been evaporated to dryness was taken up in mobile phase.

### LC-MS Analysis

The LC part was carried out on a commercially available LCMS system from Agilent Technologies, USA. For polar extracts 10 μl are injected into the system at a flow rate of 200 μl/min. The separation column (Reversed Phase C18) was maintained at 15° C. during chromatography. For lipid extracts 5 μl are injected into the system at a flow rate of 200 μl/min. The separation column (Reversed Phase C18) was maintained at 30° C. HPLC was performed with gradient elution.

The mass spectrometric analysis was performed on a Applied Biosystems API 4000 triple quadrupole instrument with turbo ion spray source. For polar extracts the instrument measures in negative ion mode in fullscan mode from 100-1000 amu. For lipid extracts the instrument measures in positive ion mode in fullscan mode from 100-1000 amu

### Derivatization of the Lipid Phase for the GC/MS Analysis

For the transmethanolysis, a mixture of 140 μl of chloroform, 37 μl of hydrochloric acid (37% by weight HCl in water), 320 μl of methanol and 20 μl of toluene was added to the evaporated extract. The vessel was sealed tightly and heated for 2 hours at 100° C., with shaking. The solution was subsequently evaporated to dryness. The residue was dried completely.

The methoximation of the carbonyl groups was carried out by reaction with methoxyamine hydrochloride (5 mg/ml in pyridine, 100 μl for 1.5 hours at 60° C.) in a tightly sealed vessel. 20 μl of a solution of odd-numbered, straight-chain fatty acids (solution of each 0.3 mg/ml of fatty acids from 7 to 25 carbon atoms and each 0.6 mg/ml of fatty acids with 27, 29 and 31 carbon atoms in 3/7 (v/v) pyridine/toluene) were added as time standards. Finally, the derivatization with 100 μl of N-methyl-N-(trimethylsilyl)-2,2,2-trifluoroacetamide (MSTFA) was carried out for 30 minutes at 60° C., again in the tightly sealed vessel. The final volume before injection into the GC was 220 μl.

### Derivatization of the Polar Phase for the GC/MS Analysis

The methoximation of the carbonyl groups was carried out by reaction with methoxyamine hydrochloride (5 mg/ml in pyridine, 50 μl for 1.5 hours at 60° C.) in a tightly sealed vessel. 10 μl of a solution of odd-numbered, straight-chain fatty acids (solution of each 0.3 mg/ml of fatty acids from 7 to 25 carbon atoms and each 0.6 mg/ml of fatty acids with 27, 29 and 31 carbon atoms in 3/7 (v/v) pyridine/toluene) were added as time standards. Finally, the derivatization with 50 μl of N-methyl-N-(trimethylsilyl)-2,2,2-trifluoroacetamide (MSTFA) was carried out for 30 minutes at 60° C., again in the tightly sealed vessel. The final volume before injection into the GC was 110 μl.

### GC-MS Analysis

The GC-MS systems consist of an Agilent 6890 GC coupled to an Agilent 5973 MSD. The autosamplers are CompiPal or GCPal from CTC. For the analysis usual commercial capillary separation columns (30 in×0.25 mm×0.25 μm) with different poly-methyl-siloxane stationary phases containing 0% up to 35% of aromatic moieties, depending on the analysed sample materials and fractions from the phase separation step, are used (for example: DB-1 ms, HP-5 ms, DB-XLB, DB-35 ms, Agilent Technologies). Up to 1 μL of the final volume is injected splitless and the oven temperature program is started at 70° C. and ended at 340° C. with different heating rates depending on the sample material and fraction from the phase separation step in order to achieve a sufficient chromatographic separation and number of scans within each analyte peak. Usual GC-MS standard conditions, for example constant flow with nominal 1 to 1.7 ml/min. and helium as the mobile phase gas are used. Ionisation is done by electron impact with 70 eV, scanning within a m/z range from 15 to 600 with scan rates from 2.5 to 3 scans/sec and standard tune conditions. Relative metabolite concentrations were calculated as log2 transformed ratios between samples and the median calculated for control samples. Subsequently, for each lower temperature of the 6 h and 78 h treatment, changes in metabolites relative to 20° C. control were determined by subtracting the log2 of the relative value of the 20° C. control treatment from the log2 of the relative value of a specific lower temperature (see table 2).

### Other Methods

In addition, sucrose, glucose, fructose, citrate, 2-oxoglutarateand starch were measured in the soluble and residual fractions of an ethanol-water extract (Scheible et al., 1997a, 1997b), as described in Stitt et al. (1989). Amino acids were determined in the ethanol/water extracts by HPLC as described in Geigenberger et al. (1996). Frozen material was used for extraction of phosphorylated metabolites and Acetyl Coenzyme A with perchloric acid and assayed as in Stitt et al. (1989) and Gibon et al. (2002).

### Results

The results of the mass spectrometry based measurements of metabolite concentrations are shown in Table 1, which shows log2 of relative metabolite concentration (compared to an internal standard) for a number of metabolites as well as sucrose, glucose, fructose.

The results from Table 1 can be presented as a function of the concentration of the metabolite at a standard temperature. These data are presented in Table 2, which shows log2 of the metabolite concentrations compared to the 20° C. control value.

In addition, the results for the metabolites which show a clear trend measured by non-mass spectrometry based assays are shown in Table 3, which shows the absolute metabolite concentration. Table 4 shows the data from Table 3 as log2 of the metabolite concentration relative to the concentration at 20° C.

Tables 5 and 7 show lists of metabolites and observed effects which show clear trends, when analysed by mass spectrometry. Tables 6 and 8 show similar lists for non-mass spectrometric analyses. In Tables 5 to 8, the metabolites are listed together with the effects observed in response to chilling in a chilling resistant plant. Plants in which these effects are duplicated at least in part are plants which can be expected to show resistance to chilling in accordance with the present invention.

TABLE 1

| | log2 of relative metabolite concentration | | | | | | | | | | | |
|---|---|---|---|---|---|---|---|---|---|---|---|---|
| | 20° C. - | 17° C. - | 14° C. - | 12° C. - | 0° C. - | 08° C. - | 20° C. - | 17° C. - | 14° C. - | 12° C. - | 10° C. - | 08° C. - |
| Name | 6 h | 6 h | 6 h | 6 h | 1 6 h | 6 h | 78 h | 78 h | 78 h | 78 h | 78 h | 78 h |
| Beta-apo-8′carotenal | 0.14 | 0.09 | 0.19 | 0.17 | 0.16 | 0.10 | −0.01 | 0.10 | 0.01 | −0.05 | 0.20 | 0.08 |
| Beta-Carotene | 0.03 | 0.04 | 0.00 | 0.06 | 0.06 | 0.08 | −0.17 | 0.03 | −0.03 | −0.05 | 0.12 | 0.09 |
| Cryptoxanthin | 0.47 | 0.39 | 0.41 | 0.74 | 0.76 | 0.76 | −0.02 | 0.62 | 0.44 | 0.38 | 0.80 | 0.43 |
| Lutein | 0.18 | 0.22 | 0.07 | 0.21 | 0.20 | 0.22 | 0.07 | 0.17 | 0.12 | 0.01 | 0.18 | 0.03 |
| Zeaxanthin | 0.07 | 0.18 | 0.11 | 0.30 | 0.15 | 0.24 | 0.04 | 0.15 | 0.12 | −0.02 | 0.15 | −0.04 |
| UDPGlucose | −0.19 | 0.30 | −0.08 | 0.13 | −0.23 | 0.04 | 0.32 | 0.08 | 0.38 | 0.60 | 0.75 | 0.73 |
| Sucrose | 0.32 | 0.93 | 1.34 | 1.79 | 1.61 | 2.05 | −0.10 | 0.40 | 0.66 | 1.32 | 1.50 | 1.64 |
| Fructose | −0.39 | 0.38 | 0.72 | 1.62 | 2.11 | 2.50 | −0.38 | 0.54 | 0.98 | 1.89 | 2.82 | 3.70 |
| Glucose | −0.76 | −0.16 | 0.01 | 0.76 | 0.05 | 1.50 | −0.48 | 0.18 | 0.50 | 1.62 | 3.02 | 3.25 |
| Raffinose | −2.29 | −2.36 | −2.36 | −2.47 | −2.06 | −2.36 | −2.21 | −2.08 | −1.63 | −1.33 | −0.23 | 0.97 |
| Myo-Inositol | −0.65 | −0.89 | −0.82 | −0.88 | −0.81 | −0.72 | −0.77 | −0.51 | 0.03 | 0.25 | 0.54 | 0.69 |
| Methylgalactopyranosid | 0.00 | 0.13 | 0.22 | 0.12 | 0.12 | 0.11 | 0.05 | 0.17 | 0.12 | 0.19 | 0.15 | 0.16 |
| Pyruvate | 0.11 | 0.21 | 0.52 | 0.53 | 0.37 | 0.50 | 0.11 | 0.04 | 0.15 | 0.27 | 0.56 | 0.40 |
| Succinate | −0.03 | −0.49 | −0.48 | −0.58 | −0.54 | −0.55 | −0.33 | −0.25 | −0.05 | −0.13 | −0.13 | −0.01 |
| Fumarate | −0.03 | −0.25 | −0.05 | −0.07 | −0.16 | −0.14 | −0.12 | −0.17 | −0.02 | −0.19 | −0.01 | 0.08 |
| Malate | −0.72 | −0.67 | −0.30 | −0.37 | −0.42 | −0.32 | −0.54 | −0.62 | −0.29 | −0.06 | 0.23 | 0.36 |
| Coenzyme Q10 | −0.12 | −0.24 | −0.34 | −0.15 | −0.25 | −0.13 | −0.39 | −0.21 | −0.13 | 0.03 | 0.15 | 0.15 |
| Ubichinone-45 (Coenzyme Q9) | −0.14 | −0.08 | −0.07 | 0.05 | 0.00 | 0.07 | −0.48 | −0.16 | −0.08 | −0.17 | 0.13 | 0.01 |
| Gly | 0.40 | 1.45 | 2.49 | 2.25 | 1.43 | 1.45 | 0.14 | 0.89 | 1.07 | 2.78 | 3.74 | 3.81 |
| Ser | −0.46 | −0.72 | −0.92 | −1.17 | −1.30 | −1.11 | −0.56 | −0.35 | −0.27 | −0.31 | −0.43 | −0.62 |
| Glycericacid | 0.11 | −0.12 | −0.35 | −0.58 | −0.73 | −0.46 | −0.03 | 0.07 | 0.10 | −0.60 | −1.02 | −1.28 |
| Glutamicacid | −0.33 | −0.63 | −0.73 | −0.98 | −0.99 | −0.82 | −0.63 | −0.48 | −0.07 | −0.03 | 0.12 | 0.12 |
| Glutamine | 0.01 | 0.21 | 0.62 | 0.58 | 0.33 | 0.52 | −0.07 | 0.23 | 0.44 | 0.96 | 1.79 | 1.90 |
| Asp | −0.55 | −0.61 | −0.42 | −0.49 | −0.38 | −0.31 | −0.42 | −0.38 | 0.01 | −0.04 | 0.31 | 0.40 |
| Ala | 0.50 | 0.62 | 0.64 | 0.56 | 0.57 | 1.06 | 0.29 | 0.67 | 0.79 | 1.50 | 2.00 | 2.10 |
| Proline | −0.31 | −0.47 | −0.41 | −0.65 | −0.44 | −0.48 | −0.48 | 0.06 | 1.27 | 2.44 | 3.21 | 3.51 |
| Homoser | | | | | | | | | | | | |
| Thr | 0.04 | −0.03 | −0.06 | −0.28 | −0.39 | −0.21 | −0.08 | −0.01 | 0.03 | −0.02 | 0.00 | 0.03 |
| Ile | −0.63 | −0.62 | −0.58 | −0.61 | −0.56 | −0.46 | −0.60 | −0.61 | −0.40 | −0.42 | −0.52 | −0.57 |
| Leu | −0.71 | −0.58 | −0.18 | −0.22 | −0.20 | −0.05 | −0.80 | −0.70 | −0.53 | −0.39 | −0.31 | −0.38 |
| Val | −0.01 | 0.19 | 0.45 | 0.42 | 0.34 | 0.58 | −0.27 | 0.00 | 0.31 | 0.55 | 0.73 | 0.76 |
| 2,3Dimethyl-5-phytylquinol | −0.08 | 0.33 | −0.21 | 0.39 | −0.30 | 0.18 | −0.36 | 0.43 | 0.39 | 0.32 | 0.76 | 0.89 |
| Methionine | 0.24 | 0.15 | 0.22 | 0.11 | 0.11 | 0.22 | 0.18 | 0.40 | 0.32 | 0.45 | 0.78 | 0.62 |
| ShikimicAcid | 0.28 | 0.05 | 0.15 | −0.06 | −0.18 | −0.06 | −0.01 | −0.09 | 0.03 | −0.08 | −0.15 | −0.27 |
| Phe | 0.68 | 0.87 | 1.16 | 1.06 | 0.77 | 0.77 | 0.22 | 0.51 | 0.68 | 0.98 | 1.54 | 1.43 |
| Tyrosine | −0.67 | −0.55 | −0.18 | −0.12 | −0.19 | −0.19 | −0.47 | −0.65 | −0.64 | −0.50 | 0.21 | 0.48 |
| Tryptophan | −0.19 | −0.04 | 0.29 | 0.27 | 0.03 | 0.08 | −0.53 | −0.33 | −0.23 | 0.08 | 0.63 | 0.77 |
| Arginine | −0.11 | −0.09 | −0.09 | 0.07 | −0.51 | 0.07 | −0.36 | −0.19 | −0.23 | −0.15 | 0.10 | 0.31 |
| Citrulline | 0.55 | 0.62 | 0.59 | 0.58 | 0.17 | 0.56 | 0.23 | 0.76 | 1.19 | 1.73 | 1.85 | 1.95 |
| GABA | −0.13 | −0.02 | −0.16 | −0.24 | −0.33 | 0.37 | 0.21 | 0.43 | 0.40 | 0.57 | 0.65 | 0.64 |
| Putrescin | −0.17 | −0.33 | −0.07 | −0.21 | −0.57 | 0.27 | −0.55 | −0.09 | 0.10 | 0.77 | 1.31 | 1.36 |
| Glycerol (polarfraction) | 0.01 | 0.28 | 0.18 | −0.14 | 0.00 | −0.15 | 0.01 | 0.22 | −0.03 | 0.18 | −0.13 | −0.18 |
| Glycerol-3- | 0.07 | −0.38 | −0.20 | −0.14 | −0.33 | −0.14 | 0.00 | −0.38 | −0.49 | −0.43 | −0.28 | −0.32 |
| phosphate (polarfraction) | | | | | | | | | | | | |
| Glycerol (lipidfraction) | 0.03 | 0.04 | 0.08 | 0.09 | 0.12 | 0.06 | 0.10 | 0.18 | 0.07 | 0.11 | 0.07 | −0.02 |
| Glycerophosphat (lipidfraction) | −0.11 | −0.22 | −0.17 | −0.25 | −0.06 | −0.10 | −0.59 | −0.36 | 0.18 | −0.03 | 0.17 | 0.15 |
| C16:0 | −0.04 | −0.08 | −0.04 | −0.06 | −0.17 | −0.11 | −0.28 | −0.16 | 0.01 | 0.03 | −0.01 | 0.04 |
| 2-Hydroxy-Palmiticacid | 0.06 | 0.00 | −0.01 | −0.02 | −0.09 | 0.07 | −0.17 | −0.11 | −0.16 | −0.02 | −0.04 | −0.04 |
| HexadecadienoicAcid (C16:2) | 0.81 | 0.62 | 0.68 | 0.81 | 0.50 | 0.53 | 0.67 | 0.58 | 0.49 | 0.54 | 0.50 | 0.42 |
| HexadecatrienoicAcid (C16:3) | 0.26 | 0.26 | 0.26 | 0.28 | 0.17 | 0.25 | 0.08 | 0.26 | 0.27 | 0.31 | 0.26 | 0.26 |
| C17:0 | −0.02 | −0.03 | 0.05 | 0.16 | 0.06 | −0.13 | −0.11 | −0.09 | −0.01 | 0.01 | 0.00 | 0.02 |
| C18:0 | −0.08 | −0.07 | −0.05 | −0.09 | −0.11 | −0.09 | −0.30 | −0.22 | −0.26 | −0.13 | −0.22 | −0.32 |
| C18:cis[9,12]2 | 0.09 | 0.05 | 0.09 | 0.07 | −0.06 | 0.06 | −0.17 | −0.03 | 0.06 | 0.08 | 0.12 | 0.19 |
| C18:cis[9,12,15]3 | 0.05 | 0.00 | 0.01 | 0.01 | −0.03 | 0.02 | −0.15 | 0.02 | 0.11 | 0.17 | 0.13 | 0.13 |
| C20:1 | −0.26 | −0.11 | 0.03 | 0.06 | 0.14 | 0.06 | 0.10 | −0.21 | 0.35 | −0.10 | 0.69 | 0.07 |
| C24:0 | −0.06 | 0.00 | 0.03 | −0.10 | 0.05 | −0.08 | −0.59 | −0.29 | −0.04 | −0.01 | 0.33 | 0.27 |
| NervonicAcid (C24:1) | 0.40 | 0.25 | 0.25 | 0.33 | 0.32 | 0.39 | 0.26 | 0.37 | 0.43 | 0.59 | 0.68 | 0.64 |
| C26:0 | −0.77 | −0.84 | −0.81 | −0.59 | −0.76 | −0.57 | −1.44 | −1.06 | −0.75 | −0.74 | −0.11 | −0.15 |
| C30:0 | −0.12 | −0.19 | −0.04 | 0.01 | −0.05 | −0.15 | −0.02 | −0.08 | −0.08 | −0.14 | −0.08 | −0.14 |
| Ribonicacid | −0.76 | −0.63 | −0.82 | −1.17 | −1.02 | −0.42 | −0.81 | −0.61 | −0.33 | −0.36 | −0.24 | −0.38 |
| alpha-Tocopherol | −0.41 | −0.59 | −0.61 | −0.52 | −0.55 | −0.53 | −0.62 | −0.43 | −0.41 | −0.40 | −0.36 | −0.33 |
| b-Tocopherol | −0.29 | −0.13 | −0.06 | −0.21 | −0.61 | 0.08 | −0.39 | 0.11 | 0.83 | 0.64 | 0.52 | 0.57 |
| gamma-Tocopherol | −0.29 | −0.13 | −0.06 | −0.21 | −0.61 | 0.08 | −0.39 | 0.11 | 0.83 | 0.64 | 0.52 | 0.57 |
| beta-Sitosterol | −0.01 | −0.09 | −0.07 | −0.03 | −0.04 | 0.00 | −0.07 | −0.06 | −0.07 | −0.07 | −0.02 | −0.11 |
| Campesterol | 0.23 | 0.16 | 0.23 | 0.13 | 0.13 | 0.26 | 0.09 | 0.26 | 0.21 | 0.25 | 0.18 | 0.24 |
| DOPA | | | | | | | | | | | | |
| Ferulicacid | −0.32 | −0.55 | −0.36 | −0.49 | −0.39 | −0.41 | −0.52 | −0.39 | −0.21 | −0.27 | −0.27 | −0.32 |
| SinapicAcid | 0.33 | 0.41 | 0.58 | 0.59 | 0.38 | 0.41 | 0.25 | 0.42 | 0.53 | 0.57 | 0.86 | 0.98 |
| IsopentenylPyrophosphate | −0.10 | −0.23 | −0.56 | −0.57 | −0.69 | −0.22 | −0.23 | −0.26 | −0.25 | −0.88 | −0.47 | −0.12 |
| Phosphate | 0.55 | −0.29 | −0.28 | 0.15 | 0.03 | 0.13 | −0.03 | 0.32 | 0.39 | 0.35 | 0.80 | 0.17 |
| Anhydroglucose | −0.83 | −0.82 | −0.78 | −1.01 | −1.01 | −0.72 | −1.12 | −0.73 | −0.48 | −0.42 | −0.68 | −0.83 |

TABLE 2

| | 20° C. - | 17° C. - | 14° C. - | 12° C. - | 10° C. - | | | 17° C. - | 14° C. - | 12° C. - | 10° C. - | 08° C. - |
|---|---|---|---|---|---|---|---|---|---|---|---|---|
| | 6 h | 6 h | 6 h | 6 h | 6 h | 08° C. - 6 h | 20° C. - 78 h | 78 h | 78 h | 78 h | 78 h | 78 h |
| beta-apo-8′-Carotenal | 0.00 | −0.05 | 0.05 | 0.03 | 0.02 | −0.04 | 0.00 | 0.10 | 0.01 | −0.04 | 0.21 | 0.08 |
| beta-Carotene | 0.00 | 0.01 | −0.03 | 0.02 | 0.03 | 0.05 | 0.00 | 0.20 | 0.14 | 0.12 | 0.29 | 0.26 |
| Cryptoxanthin | 0.00 | −0.08 | −0.07 | 0.26 | 0.29 | 0.29 | 0.00 | 0.63 | 0.45 | 0.40 | 0.82 | 0.45 |
| Lutein | 0.00 | 0.04 | −0.12 | 0.03 | 0.01 | 0.04 | 0.00 | 0.10 | 0.05 | −0.07 | 0.10 | −0.04 |
| Zeaxanthin | 0.00 | 0.11 | 0.04 | 0.23 | 0.08 | 0.18 | 0.00 | 0.11 | 0.08 | −0.06 | 0.11 | −0.08 |
| UDP-Glucose | 0.00 | 0.49 | 0.11 | 0.32 | −0.05 | 0.23 | 0.00 | −0.25 | 0.06 | 0.28 | 0.43 | 0.41 |
| Sucrose | 0.00 | 0.61 | 1.01 | 1.47 | 1.29 | 1.73 | 0.00 | 0.50 | 0.76 | 1.41 | 1.59 | 1.74 |
| Fructose | 0.00 | 0.77 | 1.12 | 2.01 | 2.51 | 2.89 | 0.00 | 0.92 | 1.35 | 2.26 | 3.20 | 4.07 |
| Glucose | 0.00 | 0.60 | 0.77 | 1.52 | 1.71 | 2.26 | 0.00 | 0.66 | 0.98 | 2.11 | 3.50 | 3.73 |
| Raffinose | 0.00 | −0.06 | −0.07 | −0.18 | 0.23 | −0.07 | 0.00 | 0.13 | 0.58 | 0.88 | 1.98 | 3.18 |
| Inositol | 0.00 | −0.24 | −0.17 | −0.23 | −0.16 | −0.07 | 0.00 | 0.26 | 0.81 | 1.02 | 1.32 | 1.47 |
| Methylgalactopyranoside | 0.00 | 0.13 | 0.22 | 0.12 | 0.11 | 0.11 | 0.00 | 0.12 | 0.07 | 0.14 | 0.10 | 0.11 |
| Pyruvate | 0.00 | 0.11 | 0.42 | 0.42 | 0.26 | 0.40 | 0.00 | −0.07 | 0.04 | 0.17 | 0.45 | 0.29 |
| Succinate | 0.00 | −0.46 | −0.46 | −0.56 | −0.51 | −0.52 | 0.00 | 0.08 | 0.28 | 0.20 | 0.20 | 0.32 |
| Fumarate | 0.00 | −0.22 | −0.02 | −0.04 | −0.13 | −0.11 | 0.00 | −0.06 | 0.09 | −0.07 | 0.10 | 0.19 |
| Malate | 0.00 | 0.05 | 0.42 | 0.35 | 0.30 | 0.40 | 0.00 | −0.09 | 0.25 | 0.48 | 0.77 | 0.89 |
| Coenzyme Q10 | 0.00 | −0.11 | −0.22 | −0.03 | −0.13 | −0.01 | 0.00 | 0.18 | 0.26 | 0.42 | 0.54 | 0.54 |
| Ubiquinone-45 | 0.00 | 0.05 | 0.07 | 0.19 | 0.14 | 0.21 | 0.00 | 0.32 | 0.40 | 0.31 | 0.61 | 0.49 |
| (Coenzyme Q9) | | | | | | | | | | | | |
| Glycine | 0.00 | 1.06 | 2.09 | 1.85 | 1.03 | 1.05 | 0.00 | 0.75 | 0.94 | 2.64 | 3.60 | 3.67 |
| Serine | 0.00 | −0.26 | −0.46 | −0.71 | −0.84 | −0.65 | 0.00 | 0.20 | 0.29 | 0.25 | 0.13 | −0.07 |
| Glycerate | 0.00 | −0.24 | −0.46 | −0.69 | −0.84 | −0.57 | 0.00 | 0.09 | 0.13 | −0.57 | −1.00 | −1.25 |
| Glutamate | 0.00 | −0.30 | −0.40 | −0.65 | −0.66 | −0.49 | 0.00 | 0.15 | 0.55 | 0.59 | 0.74 | 0.75 |
| Glutamine | 0.00 | 0.20 | 0.61 | 0.57 | 0.32 | 0.50 | 0.00 | 0.30 | 0.51 | 1.03 | 1.86 | 1.97 |
| Aspartate | 0.00 | −0.06 | 0.12 | 0.06 | 0.16 | 0.23 | 0.00 | 0.04 | 0.43 | 0.38 | 0.72 | 0.82 |
| Alanine | 0.00 | 0.12 | 0.14 | 0.06 | 0.07 | 0.57 | 0.00 | 0.38 | 0.50 | 1.21 | 1.71 | 1.81 |
| Proline | 0.00 | −0.16 | −0.10 | −0.33 | −0.13 | −0.16 | 0.00 | 0.54 | 1.75 | 2.92 | 3.69 | 3.99 |
| Homoserine | | | | | | | | | | | | |
| Threonine | 0.00 | −0.07 | −0.10 | −0.32 | −0.42 | −0.24 | 0.00 | 0.07 | 0.11 | 0.07 | 0.08 | 0.11 |
| Isoleucine | 0.00 | 0.01 | 0.05 | 0.02 | 0.06 | 0.16 | 0.00 | 0.00 | 0.21 | 0.19 | 0.08 | 0.03 |
| Leucine | 0.00 | 0.13 | 0.53 | 0.49 | 0.51 | 0.66 | 0.00 | 0.10 | 0.27 | 0.41 | 0.50 | 0.43 |
| Valine | 0.00 | 0.20 | 0.45 | 0.43 | 0.35 | 0.59 | 0.00 | 0.27 | 0.58 | 0.82 | 1.00 | 1.03 |
| 2,3-dimethyl-5-phytyl- | 0.00 | 0.40 | −0.13 | 0.47 | −0.23 | 0.25 | 0.00 | 0.79 | 0.75 | 0.68 | 1.12 | 1.25 |
| Quinol | | | | | | | | | | | | |
| Methionine | 0.00 | −0.09 | −0.02 | −0.13 | −0.13 | −0.02 | 0.00 | 0.22 | 0.14 | 0.27 | 0.60 | 0.44 |
| Shikimate | 0.00 | −0.23 | −0.13 | −0.34 | −0.46 | −0.33 | 0.00 | −0.07 | 0.04 | −0.06 | −0.14 | −0.26 |
| Phenylalanine | 0.00 | 0.20 | 0.48 | 0.38 | 0.09 | 0.10 | 0.00 | 0.29 | 0.47 | 0.77 | 1.32 | 1.22 |
| Tyrosine | 0.00 | 0.11 | 0.48 | 0.55 | 0.48 | 0.48 | 0.00 | −0.18 | −0.16 | −0.03 | 0.68 | 0.96 |
| Tryptophan | 0.00 | 0.15 | 0.48 | 0.46 | 0.22 | 0.27 | 0.00 | 0.21 | 0.31 | 0.61 | 1.16 | 1.31 |
| Arginine | 0.00 | 0.02 | 0.02 | 0.18 | −0.39 | 0.18 | 0.00 | 0.17 | 0.14 | 0.21 | 0.46 | 0.68 |
| Citrulline | 0.00 | 0.07 | 0.04 | 0.03 | −0.38 | 0.01 | 0.00 | 0.54 | 0.96 | 1.51 | 1.62 | 1.72 |
| GABA | 0.00 | 0.11 | −0.03 | −0.11 | −0.20 | 0.50 | 0.00 | 0.23 | 0.19 | 0.36 | 0.45 | 0.44 |
| Putrescine | 0.00 | −0.16 | 0.10 | −0.04 | −0.40 | 0.44 | 0.00 | 0.46 | 0.65 | 1.31 | 1.86 | 1.91 |
| Glycerol (polar fraction) | 0.00 | 0.27 | 0.17 | −0.15 | −0.01 | −0.16 | 0.00 | 0.21 | −0.04 | 0.16 | −0.14 | −0.20 |
| Glycerol-3-P (polar | 0.00 | −0.44 | −0.27 | −0.21 | −0.40 | −0.21 | 0.00 | −0.38 | −0.50 | −0.43 | −0.28 | −0.32 |
| fraction) | | | | | | | | | | | | |
| Glycerol (lipid fraction) | 0.00 | 0.01 | 0.05 | 0.06 | 0.09 | 0.03 | 0.00 | 0.08 | −0.04 | 0.01 | −0.04 | −0.12 |
| Glycerol-3-P (lipid | 0.00 | −0.11 | −0.06 | −0.14 | 0.05 | 0.01 | 0.00 | 0.23 | 0.77 | 0.56 | 0.76 | 0.74 |
| fraction) | | | | | | | | | | | | |
| Palmitate (C16:0) | 0.00 | −0.04 | 0.00 | −0.02 | −0.13 | −0.08 | 0.00 | 0.11 | 0.29 | 0.31 | 0.27 | 0.32 |
| 2-hydroxy-Palmitate | 0.00 | −0.05 | −0.07 | −0.07 | −0.15 | 0.01 | 0.00 | 0.06 | 0.01 | 0.15 | 0.13 | 0.13 |
| (C16:0)-OH | | | | | | | | | | | | |
| Palmitolenate (C16:2) | 0.00 | −0.19 | −0.13 | 0.00 | −0.31 | −0.28 | 0.00 | −0.09 | −0.18 | −0.13 | −0.17 | −0.25 |
| Hexadecatrienoate | 0.00 | 0.00 | 0.00 | 0.03 | −0.09 | −0.01 | 0.00 | 0.18 | 0.19 | 0.23 | 0.18 | 0.17 |
| (C16:3) | | | | | | | | | | | | |
| Heptadecanoate (C17:0) | 0.00 | −0.01 | 0.07 | 0.17 | 0.07 | −0.11 | 0.00 | 0.02 | 0.10 | 0.12 | 0.11 | 0.13 |
| Stearate (C18:0) | 0.00 | 0.00 | 0.02 | −0.01 | −0.03 | −0.01 | 0.00 | 0.08 | 0.04 | 0.17 | 0.08 | −0.01 |
| Linoleate | 0.00 | −0.04 | 0.01 | −0.02 | −0.15 | −0.03 | 0.00 | 0.14 | 0.23 | 0.25 | 0.30 | 0.36 |
| (C18:cis[9,12]2) | | | | | | | | | | | | |
| Linolenate | 0.00 | −0.04 | −0.03 | −0.03 | −0.08 | −0.03 | 0.00 | 0.17 | 0.26 | 0.33 | 0.28 | 0.28 |
| (C18:cis[9,12,15]3) | | | | | | | | | | | | |
| Eicosanoate (C20:1) | 0.00 | 0.15 | 0.28 | 0.32 | 0.39 | 0.31 | 0.00 | −0.31 | 0.25 | −0.20 | 0.59 | −0.03 |
| Lignocerate (C24:0) | 0.00 | 0.06 | 0.09 | −0.04 | 0.11 | −0.02 | 0.00 | 0.30 | 0.55 | 0.58 | 0.92 | 0.85 |
| Nervonate (C24:1) | 0.00 | −0.15 | −0.15 | −0.07 | −0.09 | −0.01 | 0.00 | 0.11 | 0.17 | 0.34 | 0.42 | 0.38 |
| Hexacosanoate (C26:0) | 0.00 | −0.07 | −0.04 | 0.18 | 0.01 | 0.20 | 0.00 | 0.38 | 0.69 | 0.70 | 1.33 | 1.29 |
| Melissate (C30:0) | 0.00 | −0.08 | 0.08 | 0.13 | 0.06 | −0.03 | 0.00 | −0.06 | −0.06 | −0.12 | −0.05 | −0.12 |
| Ribonate | 0.00 | 0.13 | −0.06 | −0.40 | −0.25 | 0.34 | 0.00 | 0.20 | 0.48 | 0.45 | 0.57 | 0.43 |
| alpha-Tocopherol | 0.00 | −0.18 | −0.20 | −0.11 | −0.14 | −0.12 | 0.00 | 0.19 | 0.22 | 0.22 | 0.26 | 0.29 |
| β-tocopherol | 0.00 | 0.17 | 0.23 | 0.08 | −0.31 | 0.37 | 0.00 | 0.50 | 1.22 | 1.02 | 0.91 | 0.95 |
| gamma-Tocopherol | 0.00 | 0.17 | 0.23 | 0.08 | −0.31 | 0.37 | 0.00 | 0.50 | 1.22 | 1.02 | 0.91 | 0.95 |
| beta-Sitosterol | 0.00 | −0.08 | −0.06 | −0.02 | −0.03 | 0.00 | 0.00 | 0.01 | 0.01 | 0.00 | 0.05 | −0.04 |
| Campesterol | 0.00 | −0.07 | 0.00 | −0.10 | −0.10 | 0.02 | 0.00 | 0.17 | 0.12 | 0.17 | 0.10 | 0.16 |
| DOPA | | | | | | | | | | | | |
| Ferulate | 0.00 | −0.22 | −0.04 | −0.17 | −0.06 | −0.08 | 0.00 | 0.13 | 0.31 | 0.25 | 0.25 | 0.20 |
| Sinapinate | 0.00 | 0.08 | 0.25 | 0.26 | 0.05 | 0.08 | 0.00 | 0.17 | 0.28 | 0.31 | 0.61 | 0.73 |
| Isopentenyl | | | | | | | | | | | | |
| Pyrophosphate | 0.00 | −0.13 | −0.46 | −0.47 | −0.59 | −0.12 | 0.00 | −0.03 | −0.02 | −0.65 | −0.24 | 0.11 |
| Phosphate | 0.00 | −0.84 | −0.83 | −0.40 | −0.52 | −0.42 | 0.00 | 0.35 | 0.42 | 0.38 | 0.83 | 0.20 |
| Anhydroglucose | 0.00 | 0.01 | 0.05 | −0.18 | −0.18 | 0.11 | 0.00 | 0.39 | 0.64 | 0.70 | 0.44 | 0.29 |

TABLE 3

| | | 6 h | | | | | | 78 h | | | | | |
|---|---|---|---|---|---|---|---|---|---|---|---|---|---|
| | | 20° C. | 17° C. | 14° C. | 12° C. | 10° C. | 08° C. | 20° C. | 17° C. | 14° C. | 12° C. | 10° C. | 08° C. |
| Glucose | μmol_g-1FW | 1.19 | 1.30 | 1.59 | 2.41 | 2.44 | 3.06 | 1.66 | 1.54 | 2.83 | 3.77 | 5.94 | 9.54 |
| Fructose | μmol_g-1FW | 1.65 | 1.36 | 1.62 | 2.04 | 2.41 | 2.31 | 1.53 | 2.18 | 2.01 | 2.02 | 2.74 | 3.67 |
| Sucrose | μmol_g-1FW | 2.67 | 3.45 | 4.76 | 6.93 | 6.71 | 8.46 | 3.07 | 2.76 | 4.00 | 4.93 | 6.87 | 8.33 |
| Glutamate | μmol_g-1FW | 3.68 | 2.96 | 2.25 | 2.08 | 2.49 | 2.78 | 3.70 | 4.05 | 5.10 | 5.97 | 5.50 | 5.36 |
| Aspartate | μmol_g-1FW | 1.64 | 2.04 | 1.89 | 1.70 | 1.57 | 1.69 | 1.45 | 1.64 | 1.84 | 2.13 | 2.15 | 2.38 |
| Asparagine | μmol_g-1FW | 0.50 | 0.60 | 0.58 | 0.61 | 0.52 | 0.52 | 0.68 | 0.67 | 0.67 | 0.84 | 1.13 | 1.17 |
| Serine | μmol_g-1FW | 1.27 | 1.15 | 0.97 | 0.82 | 0.77 | 0.78 | 1.38 | 1.39 | 1.57 | 1.65 | 1.50 | 1.39 |
| Glutamine | μmol_g-1FW | 2.07 | 3.39 | 4.52 | 4.68 | 3.93 | 3.87 | 2.63 | 3.12 | 3.68 | 5.73 | 10.23 | 10.67 |
| Glycine | μmol_g-1FW | 0.55 | 1.12 | 2.17 | 2.07 | 1.49 | 1.27 | 0.48 | 0.75 | 1.06 | 3.00 | 6.68 | 6.11 |
| Threonine | μmol_g-1FW | 0.72 | 0.80 | 0.85 | 0.72 | 0.71 | 0.71 | 0.86 | 0.79 | 0.91 | 0.92 | 0.96 | 1.04 |
| Citrulline | μmol_g-1FW | 0.13 | 0.13 | 0.11 | 0.13 | 0.10 | 0.11 | 0.11 | 0.16 | 0.22 | 0.34 | 0.36 | 0.38 |
| Alanine | μmol_g-1FW | 0.82 | 0.87 | 0.84 | 0.87 | 0.95 | 1.15 | 0.85 | 0.88 | 1.12 | 1.79 | 2.38 | 2.47 |
| Arginine | μmol_g-1FW | 0.07 | 0.09 | 0.09 | 0.13 | 0.09 | 0.13 | 0.09 | 0.10 | 0.10 | 0.10 | 0.14 | 0.18 |
| Valine | μmol_g-1FW | 0.10 | 0.12 | 0.16 | 0.17 | 0.17 | 0.16 | 0.12 | 0.12 | 0.13 | 0.18 | 0.19 | 0.19 |
| Leucine | μmol_g-1FW | 0.03 | 0.03 | 0.04 | 0.04 | 0.04 | 0.04 | 0.03 | 0.03 | 0.03 | 0.04 | 0.04 | 0.04 |
| G6P | μmol_g-1FW | 252.74 | 334.30 | 350.40 | 454.77 | 439.78 | 451.25 | 247.28 | 281.15 | 331.98 | 398.64 | 491.22 | 569.58 |
| Starch | μmol_g-1FW | 44.60 | 40.21 | 43.43 | 41.22 | 34.68 | 38.03 | 34.91 | 34.59 | 39.13 | 54.54 | 60.45 | 58.21 |
| Total amino acids | μmol_g-1FW | 10.53 | 13.42 | 13.64 | 12.14 | 11.32 | 11.55 | 9.77 | 10.71 | 13.25 | 18.98 | 26.35 | 33.53 |
| β-Alanine | μmol_g-1FW | 0.03 | 0.04 | 0.04 | 0.04 | 0.05 | 0.05 | 0.04 | 0.04 | 0.04 | 0.05 | 0.05 | 0.06 |
| Methionine | μmol_g-1FW | 0.05 | 0.05 | 0.05 | 0.05 | 0.08 | 0.07 | 0.10 | 0.11 | 0.09 | 0.08 | 0.08 | 0.06 |
| Norvaline | μmol_g-1FW | 0.01 | 0.01 | 0.01 | 0.01 | 0.01 | 0.01 | 0.01 | 0.01 | 0.02 | 0.01 | 0.01 | 0.01 |
| Tryptophane | μmol_g-1FW | 0.01 | 0.01 | 0.02 | 0.02 | 0.02 | 0.01 | 0.01 | 0.01 | 0.01 | 0.01 | 0.03 | 0.03 |
| Lysine | μmol_g-1FW | 0.02 | 0.02 | 0.02 | 0.02 | 0.02 | 0.02 | 0.03 | 0.02 | 0.02 | 0.02 | 0.02 | 0.01 |
| 3- | nmol_g-1FW | 461.49 | 358.46 | 603.05 | 654.04 | 619.42 | 555.73 | 381.47 | 485.01 | 513.49 | 592.73 | 612.50 | 690.85 |
| Phosphoglyceric | | | | | | | | | | | | | |
| acid | | | | | | | | | | | | | |
| PEP | nmol_g-1FW | 30.73 | 30.96 | 25.16 | 29.86 | 23.85 | 30.97 | 22.59 | 25.11 | 25.10 | 26.46 | 28.86 | 26.32 |
| Acetyl | nmol_g-1FW | 0.84 | 0.66 | 0.79 | 0.83 | 0.84 | 0.75 | 0.83 | 0.86 | 0.95 | 1.04 | 1.07 | 1.02 |
| coenzyme A | | | | | | | | | | | | | |
| Citrate | μmol_g-1FW | 3.50 | 6.05 | 5.97 | 4.54 | 7.54 | 5.17 | 5.85 | 8.74 | 8.93 | 11.10 | 11.86 | 11.64 |
| 2-oxoglutarate | nmol_g-1FW | 106.33 | 97.58 | 114.80 | 93.91 | 104.95 | 87.62 | 96.90 | 127.57 | 142.15 | 129.29 | 148.03 | 127.83 |

TABLE 4

| | 20° C. - | 17° C. - | 14° C. - | 12° C. - | 10° C. - | | | 17° C. - | 14° C. - | 12° C. - | 10° C. - | 08° C. - |
|---|---|---|---|---|---|---|---|---|---|---|---|---|
| | 6 h | 6 h | 6 h | 6 h | 6 h | 08° C. - 6 h | 20° C. - 78 h | 78 h | 78 h | 78 h | 78 h | 78 h |
| Glucose | 0.00 | 0.13 | 0.43 | 1.02 | 1.04 | 1.37 | 0.00 | −0.10 | 0.77 | 1.18 | 1.84 | 2.52 |
| Fructose | 0.00 | −0.28 | −0.02 | 0.30 | 0.55 | 0.49 | 0.00 | 0.51 | 0.39 | 0.39 | 0.84 | 1.26 |
| Sucrose | 0.00 | 0.37 | 0.83 | 1.38 | 1.33 | 1.66 | 0.00 | −0.15 | 0.38 | 0.68 | 1.16 | 1.44 |
| Glutamate | 0.00 | −0.31 | −0.71 | −0.83 | −0.56 | −0.41 | 0.00 | 0.13 | 0.46 | 0.69 | 0.57 | 0.53 |
| Aspartate | 0.00 | 0.32 | 0.21 | 0.05 | −0.06 | 0.04 | 0.00 | 0.17 | 0.34 | 0.55 | 0.57 | 0.72 |
| Asparagine | 0.00 | 0.25 | 0.20 | 0.29 | 0.05 | 0.07 | 0.00 | −0.03 | −0.02 | 0.30 | 0.73 | 0.78 |
| Serine | 0.00 | −0.15 | −0.40 | −0.64 | −0.73 | −0.71 | 0.00 | 0.01 | 0.19 | 0.26 | 0.12 | 0.02 |
| Glutamine | 0.00 | 0.71 | 1.12 | 1.17 | 0.92 | 0.90 | 0.00 | 0.24 | 0.48 | 1.12 | 1.96 | 2.02 |
| Glycine | 0.00 | 1.02 | 1.97 | 1.91 | 1.43 | 1.20 | 0.00 | 0.64 | 1.14 | 2.64 | 3.79 | 3.67 |
| Threonine | 0.00 | 0.15 | 0.23 | −0.01 | −0.04 | −0.03 | 0.00 | −0.11 | 0.08 | 0.10 | 0.17 | 0.28 |
| Citrulline | 0.00 | 0.03 | −0.17 | 0.01 | −0.40 | −0.17 | 0.00 | 0.62 | 1.06 | 1.68 | 1.75 | 1.83 |
| Alanine | 0.00 | 0.09 | 0.03 | 0.08 | 0.21 | 0.49 | 0.00 | 0.06 | 0.40 | 1.08 | 1.49 | 1.55 |
| Arginine | 0.00 | 0.33 | 0.29 | 0.77 | 0.35 | 0.83 | 0.00 | 0.17 | 0.09 | 0.16 | 0.62 | 1.01 |
| Valine | 0.00 | 0.25 | 0.62 | 0.68 | 0.68 | 0.60 | 0.00 | 0.09 | 0.18 | 0.62 | 0.74 | 0.76 |
| Leucine | 0.00 | 0.02 | 0.57 | 0.70 | 0.72 | 0.60 | 0.00 | 0.02 | 0.12 | 0.50 | 0.74 | 0.61 |
| G6P | 0.00 | 0.40 | 0.47 | 0.85 | 0.80 | 0.84 | 0.00 | 0.19 | 0.42 | 0.69 | 0.99 | 1.20 |
| Starch | 0.00 | −0.15 | −0.04 | −0.11 | −0.36 | −0.23 | 0.00 | −0.01 | 0.16 | 0.64 | 0.79 | 0.74 |
| Total amino acids | 0.00 | 0.35 | 0.37 | 0.21 | 0.10 | 0.13 | 0.00 | 0.13 | 0.44 | 0.96 | 1.43 | 1.78 |
| β-Alanine | 0.00 | 0.55 | 0.48 | 0.75 | 0.98 | 0.95 | 0.00 | 0.05 | 0.23 | 0.45 | 0.43 | 0.77 |
| Methionine | 0.00 | 0.07 | 0.01 | −0.07 | 0.60 | 0.47 | 0.00 | 0.20 | −0.06 | −0.33 | −0.35 | −0.70 |
| Norvaline | 0.00 | −0.34 | −0.09 | −0.04 | −0.34 | −0.63 | 0.00 | 0.30 | 0.84 | 0.66 | 0.74 | 0.70 |
| Tryptophane | 0.00 | 0.60 | 1.16 | 1.28 | 1.12 | 0.63 | 0.00 | −0.05 | 0.20 | 0.28 | 1.24 | 1.14 |
| Lysine | 0.00 | −0.21 | −0.17 | −0.47 | −0.21 | −0.28 | 0.00 | −0.52 | −0.46 | −0.61 | −0.72 | −0.84 |
| 3-Phosphoglyceric acid | 0.00 | −0.36 | 0.39 | 0.50 | 0.42 | 0.27 | 0.00 | 0.35 | 0.43 | 0.64 | 0.68 | 0.86 |
| PEP | 0.00 | 0.01 | −0.29 | −0.04 | −0.37 | 0.01 | 0.00 | 0.15 | 0.15 | 0.23 | 0.35 | 0.22 |
| Acetyl coenzyme A | 0.00 | −0.35 | −0.08 | −0.01 | 0.00 | −0.17 | 0.00 | 0.06 | 0.20 | 0.32 | 0.37 | 0.30 |
| Citrate | 0.00 | 0.79 | 0.77 | 0.37 | 1.11 | 0.56 | 0.00 | 0.58 | 0.61 | 0.92 | 1.02 | 0.99 |
| 2-oxoglutarate | 0.00 | −0.12 | 0.11 | −0.18 | −0.02 | −0.28 | 0.00 | 0.40 | 0.55 | 0.42 | 0.61 | 0.40 |

TABLE 5
Pyruvate - increase in concentration after short term chilling and/or increase in
concentration after medium term chilling;
Coenzyme Q9 - increase in concentration after short term chilling and/or increase in
concentration after medium term chilling;
Cerotic Acid (C26:0) - increase in concentration after medium term chilling;
β-Tocopherol - increase in concentration after medium term chilling;
γ-Tocopherol - increase in concentration after medium term chilling;
2,3-Dimethyl-5-phytylquinol - increase in concentration after medium term chilling;
Lignoceric acid (C24:0) - increase in concentration after medium term chilling;
Coenzyme Q10 - increase in concentration after medium term chilling;
Linolenic acid (C18:cis[9,12,15]3) - increase in concentration after medium term
chilling;
Heptadecanoic acid (C17:0) - increase in concentration after medium term chilling;
α-Tocopherol - increase in concentration after medium term chilling;
Linoleic acid (C18:cis[9,12]2) - increase in concentration after medium term
chilling;
Hexadecadienoic acid (C16:cis[7,10]2) - decrease in concentration after medium
term chilling;
UDPGlucose - increase in concentration after medium term chilling;
Glycerol-3-phosphate, polar fraction - decrease in concentration after medium term
chilling;
Glycerol-3-phosphate, lipid fraction - increase in concentration after medium term
chilling;
Nervonic Acid (C24:1) - increase in concentration after medium term chilling;
Glycine - increase in concentration after short term chilling and/or increase in
concentration after medium term chilling;
valine - increase in concentration after short term chilling and/or increase in
concentration after medium term chilling;
Glutamine - increase in concentration after short term chilling and/or increase in
concentration after medium term chilling;
Fructose - increase in concentration after short term chilling and/or increase in
concentration after medium term chilling;
Leucine - increase in concentration after short term chilling and/or increase in
concentration after medium term chilling;
Glucose - increase in concentration after short term chilling and/or increase in
concentration after medium term chilling;
Aspartic acid - increase in concentration after short term chilling and/or increase in
concentration after medium term chilling;
Sucrose - increase in concentration after short term chilling and/or increase in
concentration after medium term chilling;
Tyrosine - increase in concentration after short term chilling and/or increase in
concentration after medium term chilling;
Malate - increase in concentration after short term chilling and/or increase in
concentration after medium term chilling;
Glutamate - decrease in concentration after short term chilling and/or increase in
concentration after medium term chilling;
Succinate - decrease in concentration after short term chilling and/or increase in
concentration after medium term chilling;
Citrulline - increase in concentration after medium term chilling;
Arginine - increase in concentration after medium term chilling;
Proline - increase in concentration after medium term chilling;
gamma-Aminobutyric acid (GABA) - increase in concentration after medium term
chilling;
Putrescine - increase in concentration after medium term chilling;
Ribonic acid - increase in concentration after medium term chilling;
Phenylalanine - increase in concentration after medium term chilling;
Palmitic acid (C16:0) - increase in concentration after medium term chilling;
Myo-Inositol - increase in concentration after medium term chilling;
Tryptophane - increase in concentration after medium term chilling;
Sinapic Acid - increase in concentration after medium term chilling;
Raffinose - increase in concentration after medium term chilling;
Glyceric acid - decrease in concentration after short term chilling;
Shikimic Acid - decrease in concentration after short term chilling and/or decrease in
concentration after medium term chilling;
Serine - decrease in concentration after short term chilling;

TABLE 6
Glucose-6-Phosphate (G6P) - increase in concentration after short term chilling
and/or increase in concentration after medium term chilling;
β-Alanine - increase in concentration after short term chilling and/or increase in
concentration after medium term chilling;
3-Phosphoglyceric acid - increase in concentration after short term chilling and/or
increase in concentration after medium term chilling;
Methionine - increase in concentration after short term chilling and/or decrease in
concentration after medium term chilling;
Lysine - decrease in concentration after short term chilling and/or decerase in
concentration after medium term chilling;
Starch - increase in concentration after medium term chilling;
Total amino acids - increase in concentration after medium term chilling;
Norvaline - increase in concentration after medium term chilling;
Tryptophane - increase in concentration after medium term chilling;
Phosphoenolpyruvate (PEP) - increase in concentration after medium term chilling;
Acetyl Coenzyme A - increase in concentration after medium term chilling;
Citrate - increase in concentration after medium term chilling;
2-oxoglutarate - increase in concentration after medium term chilling;
Glucose - increase in concentration after short term chilling and/or increase in
concentration after medium term chilling;
Fructose - increase in concentration after short term chilling and/or increase in
concentration after medium term chilling;
Sucrose - increase in concentration after short term chilling and/or increase in
concentration after medium term chilling;
Glutamine - increase in concentration after short term chilling and/or increase in
concentration after medium term chilling;
Glycine - increase in concentration after short term chilling and/or increase in
concentration after medium term chilling;
Alanine - increase in concentration after short term chilling and/or increase in
concentration after medium term chilling;
Valine - increase in concentration after short term chilling and/or increase in
concentration after medium term chilling;
Leucine - increase in concentration after short term chilling and/or increase in
concentration after medium term chilling;
Glutamate - decrease in concentration after short term chilling and/or increase in
concentration after medium term chilling;
Serine - decrease in concentration after short term chilling;
Asparagine increase in concentration after medium term chilling;
Citrulline increase in concentration after medium term chilling;
Arginine increase in concentration after medium term chilling.

TABLE 7
Pyruvate - increase in concentration after short term chilling and/or increase in
concentration after medium term chilling;
Coenzyme Q9 - increase in concentration after short term chilling and/or increase in
concentration after medium term chilling;
Cerotic Acid (C26:0) - increase in concentration after medium term chilling;
β-Tocopherol - increase in concentration after medium term chilling;
γ-Tocopherol - increase in concentration after medium term chilling;
2,3-Dimethyl-5-phytylquinol - increase in concentration after medium term chilling;
Lignoceric acid (C24:0) - increase in concentration after medium term chilling;
Coenzyme Q10 - increase in concentration after medium term chilling;
Linolenic acid (C18:cis[9,12,15]3) - increase in concentration after medium term
chilling;
Heptadecanoic acid (C17:0) - increase in concentration after medium term chilling;
α-Tocopherol - increase in concentration after medium term chilling;
Linoleic acid (C18:cis[9,12]2) - increase in concentration after medium term
chilling;
Hexadecadienoic acid (C16:cis[7,10]2) - decrease in concentration after medium
term chilling;
UDPGlucose - increase in concentration after medium term chilling;
Glycerol-3-phosphate, polar fraction - decrease in concentration after medium term
chilling;
Glycerol-3-phosphate, lipid fraction - increase in concentration after medium term
chilling;
Nervonic Acid (C24:1) - increase in concentration after medium term chilling.

TABLE 8

G6P - increase in concentration after short term chilling and/or increase in
concentration after medium term chilling;
β-Alanine - increase in concentration after short term chilling and/or increase in
concentration after medium term chilling;
3-Phosphoglyceric acid - increase in concentration after short term chilling and/or
increase in concentration after medium term chilling;
Methionine - increase in concentration after short term chilling and/or decrease in
concentration after medium term chilling;
Lysine - decrease in concentration after short term chilling and/or decrease in
concentration after medium term chilling;
Starch - increase in concentration after medium term chilling;
Total amino acids - increase in concentration after medium term chilling;
Norvaline - increase in concentration after medium term chilling;
Tryptophane - increase in concentration after medium term chilling;
Phosphoenolpyruvate - increase in concentration after medium term chilling;
Acetyl Coenzyme A - increase in concentration after medium term chilling;
Citrate - increase in concentration after medium term chilling;
2-oxoglutarate - increase in concentration after medium term chilling.

As shown in Tables 1, 2, 3 and 4, decreased temperature affected the levels of a large number of metabolites, with many of the changes appearing in response to small changes in temperature.

Decreased temperature led within 6 h to a progressive increase of Glucose-6-Phosphate, UDP-Glucose and other phosphorylated intermediates, while Pi decreased. The increase of phosphorylated intermediates was already detectable at 17° C.

Sucrose and, to a lesser extent reducing sugars, increased and starch decreased. The vast bulk of the tissue in an Arabidopsis rosette is source leaves. The accumulation of sugars could be partly due to inhibition of phloem export. However, the decrease of starch shows that there is a shift of partitioning to favor sucrose synthesis. This response is detectable by 12° C.

There was an increase of pyruvate and malate, whereas fumarate and succinate declined slightly. This might indicate a restriction of respiration.

In central nitrogen metabolism, low temperatures led within 6 h to a small increase of Gln and Gly and decrease of Ser and Glu. The decrease of Glu is especially striking, because Glu levels are usually constant. Some minor amino acids (e.g., Phe, Tryp, Val, Leu, Thr) showed a small increase at intermediate but not at lower temperatures. There was a marked decrease of shikimate, homoserine and citrulline, three intermediates in amino biosynthesis that are detected by these profiling platforms. The responses of these three intermediates and the stabilization or decline of many minor amino acids in the lower temperature range indicate that amino acid biosynthesis is restricted by low temperatures. The response contrasts with that of sugars, which rise at low temperatures (see above). This difference is striking, because the levels of sugars and minor amino acids typically change in parallel.

A decrease of isopentyl pyrophosphate (IPP) indicated a possible block in the mevalonate pathway for terpenoid synthesis. In lipid metabolism, there was a decrease of free glycerol-3-P but no rapid change in esterified glycerol-3-P, a slight decrease of C18:1 and an increase of C26 but no other marked changes of fatty acids.

Many of these changes were maintained or strengthened after 78 h, but there were also some important modifications. There was a stronger accumulation of carbohydrates, including not only sucrose but also reducing sugars and starch (FIGS. 2-3). There was a general accumulation of organic acids, including not only pyruvate and malate but also succinate and fumarate. The initial decrease of Glu was reversed and there was a general increase of most amino acids including all the major amino acids (Gln, Glu, Asp, Ala, Asn, Gly, Ser) and many of the minor amino acids (Phe, Tryp, Val, Arg, Leu). The general accumulation of metabolites in central carbon and nitrogen metabolism may be a consequence of lower rates of export and growth. It was detectable at 14° C., and marked at 12° C. and lower temperatures.

Raffinose and proline increased markedly by 78 h. There was also an increase of other metabolites that are implicated in resistance to other abiotic stresses, including myoinositol, putrescine, α- and γ-tocopherol, putrescine, GABA, DOPA, quinol and coenzyme-Q. Myoinositol is an intermediate in the synthesis of galactinol, which is a precursor for raffinose. There was an increase of ferulic and sinapic acid, which is indicative of a stimulation of phenylpropanoid metabolism. Remarkably, many of these stress-related metabolites showed already an increase at 17° C. (e.g. raffinose, proline, putrescine, β and γ-tocopherol) and all showed marked changes by 12° C. These findings are particularly surprising at this modest temperature reduction.

There were marked changes of the lipid profile after 78 h, including an increase of esterified glycerol-3-P in the lipid fraction, an increase of unsaturated fatty acids including C18:2, C18:3 and C16:3 and a decrease of C16:2 and 16:1. There was also a small increase of some highly saturated fatty acids (including 16:0) and a marked increase of long chain unsaturated fatty acids (C24:0 and C26:0). The latter may be involved in wax formation.

Summarizing, even a small drop of the temperature leads within 6 h to unexpected marked changes in central metabolism, including accumulation of phosphorylated intermediates and decreased Pi, a shift in photoassimilate partitioning to favor sucrose rather than starch, and decreased levels of selected amino acids including Glu. After 3 days, there is a general accumulation of carbohydrates and amino acids, which may be a consequence of the decreased rates of growth. There is also an accumulation of many metabolites related to stress responses, and changes in lipid composition. Surprisingly some of these changes are already visible at 17° C. and most were detectable at 14° C. and marked at 12° C., underlining the sensitivity of the metabolic response to small changes of the temperature.

### REFERENCES

Cook D., Fowler S., Fiehn O. & Thomashow M. F. (2004) A prominent role for the CBF cold response pathway in configuring the low-temperature metabolome of Arabidopsis. Proceedings of the National Academy of Sciences USA 101, 15243-15248.
Fowler S. & Thomashow M. F. (2002) Arabidopsis transcriptome profiling indicates that multiple regulatory pathways are activated during cold acclimation in addition to the CBF cold response pathway. Plant Cell 14, 1675-1690.
Geigenberger, P., Lerchl, J., Stitt, M., Sonnewald, U. (1996) Phloem-specific expression of pyrophosphatase inhibits long-distance transport of carbohydrates and amino acids in tobacco plants. Plant, Cell Environ., 19, 43 55.
Gibon, Y., Vigeolas, H., Tiessen, A., Geigenberger, P., and Stitt, M. (2002). Sensitive and high throughput metabolite assays for inorganic pyrophosphate, ADPGlc, nucleotide phosphates, and glycolytic intermediates based on a novel enzymic cycling system. Plant J 30, 221-235.

Kreps J. A, Wu Y., Chang H. S., Zhu T., Wang X. & Harper J F. (2002) Transcriptome changes for Arabidopsis in response to salt, osmotic, and cold stress. Plant Physiology 130, 2129-2141.

Lee B. H., Henderson D. A. & Zhu J. K. (2005) The Arabidopsis cold-responsive transcriptome and its regulation by ICE1. Plant Cell 17, 3155-3175.

Provart N. J., Gil P., Chen W., Han B., Chang H. S., Wang X., & Zhu T. (2003) Gene expression phenotypes of Arabidopsis associated with sensitivity to low temperatures. Plant Physiology 132, 893 -906.

Scheible W.-R., Gonzales-Fontes A., Morcuende R., Lauerer M., Geiger M., Glaab J., Schulze E.-D. & Stitt M. (1997a) Tobacco mutants with a decreased number of functional ilia-genes compensate by modifying the diurnal regulation transcription, post-translational modification and turnover of nitrate reductase. Planta 203, 305-319.

Scheible W.-R., Gonzalez-Fontez A., Lanerer M., Müller-Röber B., Caboche M. & Stitt M. (1997b) Nitrate acts as a signal to induce organic acid metabolism and repress starch metabolism in tobacco. Plant Cell 9, 809-824.

Stitt M., Mc Lilley R. C., Gerhardt R. & Heldt H. W. (1989) Determination of metabolite levels in specific cells and subcellular compartments of plant leaves. Methods in Enzymology 174, 518-552.

Vogel J. T., Zarka D. G., Van Buskirk H. A., Fowler S. G. & Thomashow M. F. (2005) Roles of the CBF2 and ZAT12 transcription factors in configuring the low temperature transcriptome of Arabidopsis. Plant Journal 4, 195-211.

1. A method for identifying whether a plant is resistant to chilling, comprising the steps of:
a) cultivating the plant at a reduced temperature for a time period selected from short term or medium term;
b) harvesting a tissue sample of the plant;
c) measuring concentration of at least one metabolite in the tissue sample of the plant;
d) comparing the measured concentration with the concentration of the same metabolite in a tissue sample harvested from a reference plant or a plant of the same species after cultivation at a reference temperature; and
c) optionally repeating the process of steps (a) to (d) but cultivating the plant at a reduced temperature for a time period selected from short term or medium term and not previously used in step (a); wherein resistance to chilling is indicated by one or more of the effects on metabolites listed in Table 2 or Table 4.

2. The method according to claim 1, wherein resistance to chilling is indicated by measuring two or more of the effects listed in table 5 or table 6.

3. The method according to claim 2, wherein resistance to chilling is determined by mass spectrometry and indicated by measuring two or more of the effects on the metabolites set forth in Table 5.

4. The method according to claim 2, wherein resistance to chilling is determined by methods other than mass spectrometry, and indicated by measuring two or more of the effects on the metabolites set forth in Table 6.

5. The method of claim 2, wherein 3 or more of the effects on 3 or more metabolites in Table 5 or Table 6 are measured.

6. The method according to claim 1, wherein resistance to chilling is indicated by one or more of the effects listed in table 7 or table 8.

7. A The method according to claim 6, wherein resistance to chilling is determined by mass spectrometry and indicated by one or more of the effects on the metabolites set forth in Table 7.

8. The method according to claim 6, wherein resistance to chilling may be determined by methods other than mass spectrometry, and indicated by one or more of the effects on the metabolites set forth in Table 8.

9. The method according to claim 6, whereon two or more of the effects on two or more metabolites in Table 7 or Table 8 are measured on the corresponding number of metabolites.

10. The method according to claim 6, wherein resistance to chilling is indicated by one or more of the effects set forth in table 5 or Table 6 in combination with one or more of the effects set forth in Table 7 or Table 8.

11. The method of claim 1, which comprises measuring the concentration of at least five metabolites.

12. The method according to claim 11, which comprises measuring the concentrations of at least ten metabolites.

13. The method according to claim 12, which comprises measuring the concentrations of at least twenty of the metabolites.

14. The method according to claim 13, which comprises measuring the concentrations of at least thirty metabolites.

15. The method of claim 14, which comprises measuring the concentrations of at least forty metabolites.

16. The method of claim 1, which comprises measuring metabolite concentrations after both short term and medium term chilling.

17. The method of claim 1, wherein the reduced temperature is from 6° C. to 17° C.

18. The method of claim 1, wherein the plant is an Arabidopsis species,

19. A method for obtaining plants which are resistant to chilling, the method comprising:
a) cultivating a series of plants;
b) identifying those plants which have greatest resistance to chilling using the method of claim 1; and
c) crossing the chill-resistant plants identified in step (b) to obtain progeny which are resistant to chilling.
